(19) United States
(12) Patent Application Publication
(10) Pub. No.: US 2024/0082162 A1
(43) Pub. Date: Mar. 14, 2024

(54) PHARMACEUTICAL FORMULATIONS OF PILOCARPINE R-(+)-LIPOATE

(71) Applicant: Cellix Bio Private Limited, Hyderabad (IN)

(72) Inventor: Mahesh Kandula, East Godavari District, Andhra Pradesh (IN)

(73) Assignee: Cellix Bio Private Limited, Hyderabad (IN)

(21) Appl. No.: 17/928,278

(22) PCT Filed: May 25, 2021
(86) PCT No.: PCT/IB21/54507

§ 371 Date: Jul. 28, 2023

(30) Foreign Application Priority Data
May 26, 2020 (IN) 202041021891
Jun. 27, 2020 (IN) 202041027318

(51) Int. Cl. A61K 9/20; A61K 9/00; A61K 31/4178

(52) U.S. Cl. CPC A61K 9/2054; A61K 9/0053; A61K 31/4178

(57) ABSTRACT

The present invention relates to a pharmaceutical formulations and compositions comprising cholinergic agonist agents such as pilocarpine-R-(+)-lipoate or its salt, solvate, or hydrate thereof for oral administration with improved compliance, safety, and bioavailability. It further discloses methods of preparing the formulations and its use for the treatment of xerostomia, dry mouth and Sjogren's syndrome.

### FIELD OF THE INVENTION

The present invention described herein, in general, relates to pharmaceutical formulations, and in particular, relates to the pharmaceutical formulations comprising cholinergic agonists such as derivatives and salts of Pilocarpine R-(+)-lipoate.

### BACKGROUND OF THE INVENTION

Dry mouth also known as xerostomia, occurs due to reduced flow of saliva. The most common diseases causing xerostomia include Sjogren's syndrome, radiotherapy for head and neck cancer, HIV disease and others. It has been found that xerostomia is also caused from a common side effect of some prescribed medications or due to the use of different types of medications. Occurrence of xerostomia has also been seen in those who breathe through their mouths. Several other diseases are also known to cause hyposalivation, or change in saliva consistency. Normal salivary function is mediated by the muscarinic receptors. Stimulation of this receptors results in increased flow of salivary secretions.

There are several over-the-counter products that are available to aid in the management of xerostomia. These products range from saliva substitutes and stimulants to products designed to minimize dental problems.

Parasympathomimetic (cholinergic) agents are used to treat symptoms of dry mouth associated with Sjogren's syndrome and radiotherapy, to treat elevated intraocular pressure, various types of glaucoma, and to induce miosis. Pilocarpine is a well-known muscarinic cholinergic agonist and has been used to increase salivation in patients who suffer from dry mouth in a variety of different disorders. Pilocarpine belongs to a class of drugs known as cholinergic agonists. It works by stimulating certain nerves to increase the amount of saliva production. Cholinergic drugs act at acetylcholine (ACh) which is the major neurotransmitter of parasympathetic nervous system (PSNS). Cholinergic drugs mimic actions of Ach, therefore it also referred to as cholinomimetics or parasympathomimetic agents.

Commercially available medications are formulated as solutions, sprays, gels, tablets and lozenges. Pilocarpine in the form of tablets (immediate release—Salagen®) is commercially available for use in xerostomia.

International Application WO2018065831, discloses various muscarinic agonist compounds and it salts and method of synthesis of such salts.

The efficacy of existing formulation comprising pilocarpine is limited due to its adverse side effects and multiple daily dosages.

### SUMMARY OF THE INVENTION

The present invention discloses pharmaceutical formulation comprising cholinergic agonist agents such as Pilocarpine R-(+)-lipoate. This invention relates to a formulation and methods for the treatment of patients suffering from a dry mouth condition. It further discloses methods of preparing such formulation and methods of using the formulation.

In an embodiment present invention discloses a pharmaceutical composition for oral administration comprising pilocarpine R-(+)-lipoate or its polymorphs, enantiomers, isomers; and its pharmaceutically acceptable salts thereof.

In another embodiment present invention discloses a pharmaceutical composition, wherein, the pilocarpine R-(+)-lipoate is formulated in an amount of 1% to 70% w/w of the composition, along with one or more excipients in an amount of 30-99% w/w of the composition.

In an embodiment present invention discloses a pharmaceutical composition, wherein, the pilocarpine-R-(+)-lipoate is in the range of 0.1 mg to 100 mg.

In other embodiments the present invention discloses a pharmaceutical composition wherein, the excipients are selected from a diluent, a lubricant, a disintegrant, a polymer, a flavoring agent, a binder, a sweeting agent, a glidant, an antioxidant, coating material, or mixtures thereof.

In other embodiments the present invention discloses a pharmaceutical composition, wherein, the diluent is selected from lactose, spray dried lactose, lactose monohydrate, lactose hydrous, lactose anhydrous, starches, maize starches, or partially pregelatinized starches, sucrose, magnesium stearate, glucose, micro crystalline cellulose, Polyvinylpyrrolidone, mannitol, sorbitol, dibasic calcium phosphate dehydrate, calcium sulphate dehydrate, calcium carbonate, or mixtures thereof.

In other embodiments the present invention discloses a pharmaceutical composition of claim 4, wherein the disintegrant is selected from crosslinked polymer such as polyvinylpyrrolidone (cro spovidone) or crosslinked sodium carboxymethylcellulo se (croscarmellose sodium), microcrystalline cellulose (MCC), alginates or modified starches, such as sodium starch glycolate, or mixtures thereof.

In other embodiments the present invention discloses a pharmaceutical composition, wherein, the polymer is selected from carbomers, polycarbophil, pemulen polymers, starch, modified cellulose, crystalline cellulose, microcrystalline cellulose, carboxymethylcellulose, sodium, carboxymethylcellulose, an acrylic acid copolymer, methyl vinyl ether copolymer with maleic anhydride, hydroxypropyl methylcellulo se, polyglycolic acid, or mixtures thereof.

In other embodiments the present invention discloses a pharmaceutical composition, wherein, the flavoring agent is selected from clove oil, citric syrup, glycerin, rose oil, orange oil, menthol, cherry, or mixtures thereof.

In other embodiments the present invention discloses pharmaceutical composition, wherein, the binder, is selected from saccharides and their derivatives such as starches, cornstarch, cellulose, methyl cellulose and modified cellulose such as microcrystalline cellulose, cellulose ethers such as hydroxypropyl cellulose; sugar alcohols such as xylitol, sorbitol, or mannitol; protein: such as gelatin; synthetic polymers such as polyvinyl pyrrolidone (PVP), polyethylene glycol (PEG), or mixtures thereof.

In other embodiments the present invention discloses a pharmaceutical composition wherein the sweeting agent is selected from sucrose, liquid glucose, glycerol, sorbitol, saccharin sodium, aspartame, or mixtures thereof.

In other embodiment the present invention discloses a pharmaceutical composition wherein, the glidant is selected from magnesium stearate, fumed silica (colloidal silicon dioxide), starch, talc, or mixtures thereof.

In other embodiments the present invention discloses a pharmaceutical composition, wherein the antioxidant is selected from butylated hydroxyanisole, butylated hydroxytoluene, sodium metabisulfite (SMB), propyl gallate (PG) cysteine (CYS), ascorbic acid, or mixtures thereof.

In other embodiments the present invention discloses a pharmaceutical composition, wherein, the a coating material is selected from sugar, polymers, polysaccharides, moisture barrier coating material, cellulosic polymers, vinyl derivatives, hydroxypropyl cellulose, Hydroxyethyl cellulose microcrystalline cellulose, derivatives cellulose, alkylated cellulose, ethyl cellulose, propyl cellulose, hydroxylpropyl cellulose, sugar or a polysaccharide, hydroxypropyl methylcellulose, carboxymethylcellulose, maltodextrin, sucrose, modified starch, a salt of alginic acid, soluble gums, carrageenan, polymer comprises polyvinylpyrrolidone or polyvinylpolypyrrolidone, and Opadry film coating system.

In other embodiments the present invention discloses a pharmaceutical composition, wherein, the coating of the composition is from sugar coatings, film coatings, gelatin coatings, enteric coatings, compression coatings, or immediate-release film coatings.

In other embodiments the present invention discloses a pharmaceutical composition, is a modified release composition wherein the modified release composition is formulated into mucoadhesive buccal tablet, lozenge, oral patch, oral film, buccal patch, oral spray, oral solution, oral gel, sub-lingual tablet, mucoadhesive patch or film or transdermal patch.

In other embodiments the present invention discloses pharmaceutical composition, wherein, the modified release is controlled by polymers and pharmaceutically acceptable excipients to deliver extended release, sustained release, or delayed release.

### DETAILED DESCRIPTION OF THE INVENTION

The present invention discloses pharmaceutical formulations comprising a muscarinic agonist. In an embodiment, the pilocarpine-R-(+)-lipoate (CLX 156) is formulated in solid, liquid or semi-solid formulation for immediate release or buccal action formulation or any other modified release dosage forms.

In some embodiments, the concentration of the pilocarpine-R-(+)-lipoate is in the range of 0.1 mg to 100 mg.

In another embodiment, the pilocarpine-R-(+)-lipoate is formulated for oral, topical, buccal or local administration.

In certain embodiment the buccal formulation can be a buccal adhesive tablets, patches, films, semisolids (ointments and gels), powders thereof

In some embodiments, the pilocarpine-R-(+)-lipoate is formulated in dosage forms which include tablets (peroral & chewable), capsules, bilayer tablets, pills, solutions, sprays, gargles, lozenges, films, oral patches, buccal patches, transdermal patches, mouthwash products, suspensions, muco-adhesive gels, flavored chewing gums, buccal and sublingual tablets or buccal adhesive gels. For all oral and local administration, the bitter taste of the pilocarpine-R-(+)-lipoate is masked.

In some embodiments, present invention discloses a pharmaceutical formulation in a suitable dosage form, wherein the formulation comprises pilocarpine-R-(+)-lipoate is having the following chemical structure:

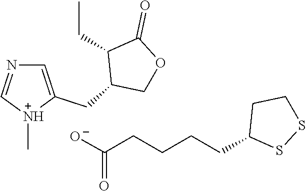

### Pilocarpine-R-(+)-lipoate (CLX 156)

or pharmaceutically acceptable forms thereof; along with pharmaceutical acceptable excipients or carries.

In some embodiments, pharmaceutical formulations comprise the Pilocarpine-R-(+)-lipoate, or a pharmaceutically acceptable forms thereof, the said formulation can be a single/multiple administrable dose to a subject.

In some embodiments, the pilocarpine-R-(+)-lipoate, or a pharmaceutically acceptable forms thereof, (i.e., the active pharmaceutical ingredient, or the API) is present in the 0.1% to about 70% by weight of the formulation.

In some embodiments, a single administrable dose for pilocarpine-R-(+)-lipoate, or a pharmaceutically acceptable form thereof, is between 0.1-70 mg. In certain embodiments, a single administrable dose of pilocarpine-R-(+)-lipoate, or a pharmaceutically acceptable form thereof.

As per present invention, the pharmaceutically acceptable excipient/carrier of pilocarpine-R-(+)-lipoate and cholinergic agonists includes inert additives such as fillers, binders, disintegrants, coatings, sorbents, anti-adherent, mucoadhesive polymers, wetting agents, glidants, lubricants, preservatives, antioxidants, flavoring agents, sweeting agents, coloring agents, solvent and co-solvent, buffering agents, chelating agents, viscosity imparting agents, surface active agents, humectants, coating material and packing materials.

In some embodiments non-limiting example of mucoadhesive polymers includes carbomers, Carbopol® 974P, polycarbophil, pemulen polymers, starch, modified cellulose, crystalline cellulose, microcrystalline cellulose, carboxymethyl cellulose, sodium carboxymethylcellulose (CMC), hydroxypropyl methylcellulose (HPMC), an acrylic acid copolymer, polyacrylic acid, methyl vinyl ether copolymer with maleic anhydride, polyglycolic acid, and others known in the art.

In some embodiments, the non-limiting example of filler and diluent includes lactose, spray dried lactose, lactose monohydrate, lactose hydrous, lactose anhydrous, starches, maize starches, or partially pregelatinized starches, sucrose, magnesium stearate, glucose, micro crystalline cellulose (Avicel® 101 and 102), Ceolus™ PH-102, Pharmacel® 112, Polyvinylpyrrolidone (PVP K30), mannitol (Pearlitol SD200 and 25C), sorbitol, dibasic calcium phosphate dihydrate, calcium sulphate dihydrate, calcium carbonate and others known in the art.

In some embodiments, the non-limiting example of disintegrants include crosslinked polymer such as polyvinylpyrrolidone (crospovidone) or crosslinked sodium carboxymethylcellulose (croscarmellose sodium), Microcrystalline cellulose (MCC), Alginates or modified starches, for example sodium starch glycolate and others known in art.

In some embodiments, the non-limiting example of nonionic, anionic, cationic, amphoteric wetting agents includes sodium lauryl sulfate, Glyceryl Monostearate, Sodium oleate, Sorbitan esters, Polyoxyethylene sorbitan esters, Triethanolamine oleate and others known in art.

In some embodiments, the non-limiting example of glidants includes magnesium stearate, fumed silica (colloidal silicon dioxide), starch, talc and others known in art.

In some embodiments, the non-limiting example of lubricant includes stearic acid, sterotex, glyceryl behenate (compritol 888), sodium stearyl fumarate, silica, hydrated magnesium silicate, sodium benzoate, sodium acetate, carbowax(PEG) 4000/6000 and others known in the art.

In some embodiments, coating is used and the coating is not limited to but includes sugar coated, film coated, gelatin coated, enteric coated, compression coated, immediate-release film coating and other know coating forms in the art. Non-limiting example of coating material includes sugar, polymers, polysaccharides, moisture barrier coating material, cellulosic polymers, vinyl derivatives, hydroxypropyl cellulose, Hydroxyethyl cellulose microcrystalline cellulose, derivatives cellulose, alkylated cellulose, ethyl cellulose, propyl cellulose, hydroxylpropyl cellulose, sugar or a polysaccharide, hydroxypropyl methylcellulose (HPMC), carboxymethylcellulose, maltodextrin, sucrose, modified starch, a salt of alginic acid, soluble gums, carrageenan, polymer comprises polyvinylpyrrolidone (PVP) or polyvinylpolypyrrolidone (PVPP), Opadry® film coating system and others known in art.

In some embodiments, the non-limiting example of binders includes saccharides and their derivatives example starches, cornstarch, cellulose, methyl cellulose or modified cellulose such as microcrystalline cellulose (MCC) and cellulose ethers such as hydroxypropyl cellulose (HPC); sugar alcohols for examples xylitol, sorbitol or mannitol; protein: such as gelatin; Synthetic polymers: e.g. polyvinylpyrrolidone (PVP), polyethylene glycol (PEG).

In some embodiments, the non-limiting example of sweetening agent include sucrose, liquid glucose, glycerol, Sorbitol, saccharin sodium, aspartame and other known in the art.

In some embodiments, the non-limiting example of flavoring agent includes clove oil, citric and syrup, glycerin, rose oil, orange oil, menthol, cherry and others known in the art.

In some embodiments, the non-limiting examples of chelating reagent includes disodium EDTA, ethylenediaminetetraacetic acid, citric acid, calcium disodium EDTA.

In some embodiments, the non-limiting examples of antioxidants include butylated hydroxyanisole (BHA), butylated hydroxytoluene (BHT), sodium metabisulfite (SMB), propyl gallate (PG) cysteine (CYS) and ascorbic acid

In some embodiments, the formulation is packed to prevent degradation of drug product due to moisture and oxidation. In certain embodiments HDPE Bottle, with silica gel bag and cotton plug is used.

In certain embodiments, the manufacturing process used includes, not limited to, direct compression, dry granulation, wet granulation with aqueous or non-aqueous solvents and other manufacturing procedures will be adapted.

In certain embodiments other compositions can also be selected from:

Composition 1: pilocarpine-R-(+)-lipoate (CLX 156), microcrystalline cellulose (Avicel® PH 102) stearic acid and opadry blue.

Composition 2: pilocarpine-R-(+)-lipoate (CLX 156), microcrystalline cellulose (Avicel® PH 102), croscarmellose sodium (AcDiSol SD-711), stearic acid and opadry blue.

Composition 3: pilocarpine-R-(+)-lipoate (CLX 156), microcrystalline cellulose (Avicel® PH 102), sodium starch glycolate, stearic acid and opadry blue.

Composition 4: pilocarpine-R-(+)-lipoate (CLX 156), microcrystalline cellulose (Avicel® PH 102), crospovidone (kollidon CL), stearic acid and opadry blue.

Composition 5: pilocarpine-R-(+)-lipoate (CLX 156), microcrystalline cellulose (Avicel® PH 102), methocel K15M CR, cherry flavor and stearic acid.

Composition 6: pilocarpine-R-(+)-lipoate (CLX 156), microcrystalline cellulose (Avicel® PH 102), sodium carboxymethylcellulose (Na. CMC), aspartame, cherry flavor and stearic acid.

Composition 7: pilocarpine-R-(+)-lipoate (CLX 156), microcrystalline cellulose (Avicel® PH 102), methocel K15M CR, aspartame, cherry flavor and stearic acid.

Composition 8: pilocarpine-R-(+)-lipoate (CLX 156), microcrystalline cellulose (Pharmacel® PH 112), Na. CMC, aspartame, cherry flavor and stearic acid.

Composition 9: pilocarpine-R-(+)-lipoate (CLX 156), Pharmacel PH 112, Na. CMC, aspartame, cherry flavor, stearic acid and anti-oxidants such as butylated hydroxyanisole (BHA) and butylated hydroxytoluene (BHT).

Composition 10: pilocarpine-R-(+)-lipoate (CLX 156), microcrystalline cellulose (Avicel® PH 102), Hydroxypropyl methylcellulose K15 CR (HPMC K15 CR), aspartame, cherry flavor and stearic acid.

Composition 11: pilocarpine-R-(+)-lipoate (CLX 156), Pharmacel PH 112, Hydroxypropyl methylcellulose K15 CR (HPMC K15 CR), aspartame, cherry flavor and stearic acid.

Composition 12: pilocarpine-R-(+)-lipoate (CLX 156), Pharmacel PH 112, Na. CMC, polycarbophil, HPMC K15 CR, aspartame, cherry flavor, stearic acid and antioxidants (BHA and BHT).

Composition 13: pilocarpine-R-(+)-lipoate (CLX 156), HPMC, corn starch, lactose monohydrate, silica, polycarbophil, carbomer, talc and magnesium stearate

In an embodiment the compositions disclosed in examples 1-17, 20-26 of the present invention can be prepared by following method in general.

### Manufacturing Procedure

1. Raw materials are dispensed using a calibrated weighing balance.
2. API and excipients except lubricant are sifted and mixed
3. Lubricant is added to above blend and mixed.
4. Powder blend compressed into tablets using suitable tooling on a compression machine.
5. Tablets are film coated

### Example 1: Composition 1

| Ingredients | mg/unit | % w/w |
|---|---|---|
| CLX 156 | 12.75 | 8.50 |
| Microcrystalline cellulose(Avicel PH-102) | 132.75 | 88.50 |
| Stearic acid | 4.50 | 3.00 |
| Uncoated Tablet Weight (mg) | 150.0 | 100.0 |

### Example 2: Composition 2

| Ingredients | mg/unit | % w/w |
|---|---|---|
| CLX 156 | 12.75 | 8.28 |
| Microcrystalline cellulose(Avicel PH-102) | 134.25 | 87.18 |
| Stearic acid | 3.00 | 1.95 |
| Uncoated Tablet Weight (mg) | 150.00 | 97.40 |
| Opadry | 4.00 | 2.60 |
| Coated Tablet weight | 154.00 | 100 |

### Example 3: Composition 3

| Ingredients | mg/unit | % w/w |
|---|---|---|
| CLX 156 | 12.75 | 8.28 |
| Microcrystalline cellulose(Avicel PH-102) | 131.25 | 85.23 |
| Croscarmellose Sodium (ACDiSol SD-711) | 3.00 | 1.95 |
| Stearic acid (Kolliwax S Fine) | 3.00 | 1.95 |
| Uncoated Tablet Weight (mg) | 150.00 | 97.40 |
| Opadry | 4.00 | 2.60 |
| Coated Tablet weight | 154.00 | 100 |

### Example 4: Composition 4

| Ingredients | Mg/unit | % w/w |
|---|---|---|
| CLX 156 | 12.75 | 8.28 |
| Microcrystalline cellulose (Avicel PH-102) | 131.25 | 85.23 |
| Croscarmellose Sodium (ACDiSol SD-711) | 3.00 | 1.95 |
| Stearic acid (Kolliwax S Fine) | 3.00 | 1.95 |
| Uncoated Tablet Weight (mg) | 150.00 | 97.40 |
| Opadry | 4.00 | 2.60 |
| Coated Tablet weight | 154.00 | 100 |

### Example 5: Composition 5

| Ingredients | Mg/unit | % w/w |
|---|---|---|
| CLX 156 | 12.75 | 8.28 |
| Microcrystalline cellulose(Avicel PH-102) | 131.25 | 85.23 |
| Sodium Starch Glycolate | 3.00 | 1.95 |
| Stearic acid (Kolliwax S Fine) | 3.00 | 1.95 |
| Uncoated Tablet Weight (mg) | 150.00 | 97.40 |
| Opadry | 4.00 | 2.60 |
| Coated Tablet weight | 154.00 | 100 |

### Example 6: Composition 6

| Ingredients | Mg/unit | % w/w |
|---|---|---|
| CLX 156 | 12.75 | 8.28 |
| Microcrystalline cellulose(Avicel PH-102) | 131.25 | 85.23 |
| Crospovidone(kollidon CL) | 3.00 | 1.95 |
| Stearic acid (Kolliwax S Fine) | 3.00 | 1.95 |
| Uncoated Tablet Weight (mg) | 150.00 | 97.40 |
| Opadry | 4.00 | 2.60 |
| Coated Tablet weight | 154.00 | 100 |

Buccal Modified Release Tablets : Examples 7 to 26

### Example 7: Composition 7

| Ingredients | mg/unit | % w/w |
|---|---|---|
| CLX 156 | 12.75 | 8.50 |
| Microcrystalline Cellulose (Accel PH-102) | 125.25 | 83.50 |
| Polyvinyl Pyrrolidone | 3.75 | 2.50 |
| Xanthan Gum | 5.00 | 3.33 |
| Cherry Flavor | 0.25 | 0.17 |
| Stearic acid | 3.00 | 2.00 |
| Uncoated Tablet Weight (mg) | 150.00 | 100.00 |

### Example 8: Composition 8

| Ingredients | mg/unit | % w/w |
|---|---|---|
| CLX 156 | 12.75 | 8.50 |
| Microcrystalline Cellulose (Ceolus PH-102) | 110.00 | 73.33 |
| Hydroxypropyl Methylcellulose (Methocel K15M CR) | 24.00 | 16.00 |
| Cherry Flavor | 0.25 | 0.17 |
| Stearic acid | 3.00 | 2.00 |
| Uncoated Tablet Weight (mg) | 150.00 | 100.00 |

### Example 9: Composition 9

| Ingredients | mg/unit | % w/w |
|---|---|---|
| CLX 156 | 12.75 | 6.38 |
| Mannitol | 109.85 | 54.93 |
| Microcrystalline cellulose (Avicel PH-102) | 50.00 | 25.00 |
| Hydroxypropyl Methylcellulose (Methocel K15M CR) | 24.00 | 12.00 |
| Aspartame | 0.15 | 0.08 |
| Cherry Flavor | 0.25 | 0.13 |
| Stearic acid | 3.00 | 1.50 |
| Uncoated Tablet Weight (mg) | 200.00 | 100.00 |

### Example 10 : Composition 10

| Ingredients | Mg/unit | % w/w |
|---|---|---|
| CLX 156 | 4.25 | 2.83 |
| Microcrystalline cellulose (Avicel PH-102) | 92.50 | 61.67 |
| Hydroxypropyl Methylcellulose (Methocel K15M CR) | 50.00 | 33.33 |
| Cherry Flavor | 0.25 | 0.17 |
| Stearic acid | 3.00 | 2.00 |
| Uncoated Tablet Weight (mg) | 150 | 100 |

### Example 11: Composition 11

| Ingredients | mg/unit | % w/w |
|---|---|---|
| CLX 156 | 12.75 | 8.50 |
| Microcrystalline cellulose (Avicel PH-102) | 83.85 | 55.90 |
| sodium carboxymethylcellulose (Blanose 7H4XF) | 50.00 | 33.33 |
| Aspartame | 0.15 | 0.10 |
| Cherry Flavor | 0.25 | 0.17 |
| Stearic acid | 3.00 | 2.00 |
| Uncoated Tablet Weight (mg) | 150.00 | 100.00 |

### Example 12 (a): Composition 12a

| Ingredients | Mg/unit | % w/w |
|---|---|---|
| CLX 156 | 12.75 | 8.50 |
| Microcrystalline cellulose (Avicel PH-102) | 83.85 | 55.90 |
| Sodium CMC | 50.00 | 33.33 |
| Aspartame | 0.15 | 0.10 |
| Cherry Flavor | 0.25 | 0.17 |
| Stearic acid | 3.00 | 2.00 |
| Uncoated Tablet Weight (mg) | 150 | 100 |

### Example 12(b) Composition 12b

| Ingredients | mg/unit | % w/w |
|---|---|---|
| CLX 156 | 12.75 | 8.50 |
| Microcrystalline cellulose (Avicel PH-102) | 58.85 | 39.23 |
| Sodium CMC | 75.00 | 50.00 |
| Aspartame | 0.15 | 0.10 |
| Cherry Flavor | 0.25 | 0.17 |
| Stearic acid | 3.00 | 2.00 |
| Uncoated Tablet Weight (mg) | 150.00 | 100.00 |

### Example 13: Composition 13

| Ingredients | mg/unit | % w/w |
|---|---|---|
| CLX 156 | 12.75 | 8.52 |
| Microcrystalline cellulose (Avicel PH-102) | 103.85 | 69.21 |
| Sodium CMC | 30.00 | 20.00 |
| Aspartame | 0.15 | 0.10 |
| Cherry Flavor | 0.25 | 0.17 |
| Stearic acid | 3.00 | 2.00 |
| Uncoated Core Tablet Weight (mg) | 150.00 | 100.00 |

### Example 14: Composition 14

| Ingredients | mg/unit | % w/w |
|---|---|---|
| CLX 156 | 8.50 | 4.23 |
| Microcrystalline cellulose (Avicel PH-102) | 98.10 | 48.81 |
| Carbomer (Carbopol 974P) | 90.00 | 44.78 |
| Aspartame | 0.15 | 0.07 |
| Cherry Flavor | 0.25 | 0.12 |
| Colloidal Silicon dioxide | 1.00 | 0.50 |
| Stearic acid | 3.00 | 1.49 |
| Uncoated Tablet Weight (mg) | 201.00 | 100.00 |

### Example 15: Composition 15

| Ingredients | mg/unit | % w/w |
|---|---|---|
| CLX 156 | 12.75 | 8.50 |
| Microcrystalline cellulose (Avicel PH-102) | 109.85 | 73.23 |
| Hydroxypropyl-Methylcellulose (Benecel K100M CR) | 24.00 | 16.00 |
| Aspartame | 0.15 | 0.10 |
| Cherry Flavor | 0.25 | 0.17 |
| Stearic acid | 3.00 | 2.00 |
| Uncoated Tablet Weight (mg) | 150.00 | 100.00 |

### Example 16: Composition 16

The composition 16 was manufactured using Methocel K100M CR as polymer at 2 different hardness 2 — 3 Kp & 5 — 6 Kp.

| Ingredients | mg/unit | % w/w |
|---|---|---|
| CLX 156 | 12.75 | 7.29 |
| Microcrystalline cellulose (Avicel PH-102) | 58.85 | 33.63 |
| Hydroxypropyl-Methylcellulose (Benecel K100M CR) | 100.00 | 57.14 |
| Aspartame | 0.15 | 0.09 |
| Cherry Flavor | 0.25 | 0.14 |
| Stearic acid | 3.00 | 1.71 |
| Uncoated Tablet Weight (mg) | 175.00 | 100.00 |

### Example 17: Composition 17

The composition 17 was manufactured using Methocel K15M CR as polymer at 2 different hardness 2-3 Kp & 5-6 Kp.

| Ingredients | mg/unit | % w/w |
|---|---|---|
| CLX 156 | 12.75 | 7.29 |
| Microcrystalline cellulose (Avicel PH-102) | 58.85 | 33.63 |
| Hydroxypropyl Methylcellulose (Methocel K15M CR) | 100.00 | 57.14 |
| Aspartame | 0.15 | 0.09 |
| Cherry Flavor | 0.25 | 0.14 |
| Stearic acid | 3.00 | 1.71 |
| Uncoated Tablet Weight (mg) | 175.00 | 100.00 |

### Example 18: Composition 18

The composition 18 was manufactured using Sodium Carboxymethyl Cellulose as polymer using Dry Granulation technique.

| Ingredients | mg/unit | % w/w |
|---|---|---|
| CLX 156 | 12.75 | 8.50 |
| Microcrystalline cellulose (Avicel PH-102) | 40.00 | 26.67 |
| Sodium Carboxymethylcellulose (Blanose CMC | 75.00 | 50.00 |
| 7H4XF) | | |
| Avicel PH-102 | 18.85 | 12.57 |
| Aspartame | 0.15 | 0.10 |
| Cherry Flavor | 0.25 | 0.17 |
| Stearic acid | 3.00 | 2.00 |
| Uncoated Tablet Weight (mg) | 150.00 | 100.00 |

### Manufacturing Procedure for example 18

1. Raw materials were dispensed using a calibrated weighing balance.

2. API, part Avicel PH-102 & Sodium CMC were sifted and mixed.

3. The powder blend was compressed into slugs using compression machine.

4. The obtained slugs were milled and sifted using #20 ASTM sieve. Fines were re-slugged and milled.

5. The obtained final granules were mixed with extra granular materials (part Avicel PH-102 & Aspartame).

6. Stearic Acid was added to above blend and mixed.

7. Powder blend compressed to tablets using compression machine and 8 mm round tooling.

### Example 19: Composition 19

The composition 19 was manufactured using Sodium Carboxymethyl Cellulose as polymer using Wet Granulation technique.

| Ingredients | mg/unit | % w/w |
|---|---|---|
| CLX 156 | 12.75 | 8.50 |
| Microcrystalline cellulose (Avicel PH-102) | 65.00 | 43.33 |
| Sodium carboxymethylcellulose (Blanose CMC | 50.00 | 33.33 |
| 7H4XF) | | |
| Isopropyl Alcohol | Q.S. | Q.S |
| Avicel PH-102 | 18.85 | 12.57 |
| Aspartame | 0.15 | 0.10 |
| Cherry Flavor | 0.25 | 0.17 |
| Stearic acid | 3.00 | 2.00 |
| Uncoated Tablet Weight (mg) | 150.00 | 100.00 |

Manufacturing Procedure for example 19
1. Raw materials were dispensed using a calibrated weighing balance
2. API, Avicel PH-102 & Sodium CMC were sifted and mixed
3. The powder blend was granulated with Isopropyl Alcohol.
4. The wet mass was dried and the dried granules were sifted through #24 ASTM sieve.
5. Extra granular materials were added and mixed, Stearic Acid was blended and Powder blend compressed to tablets using compression machine and 8 mm round.

### Example 20: Composition 20

The composition 20 was manufactured using Polycarbophil as polymer, Dry mixing technique.

| Ingredients | mg/unit | % w/w |
|---|---|---|
| CLX 156 | 12.75 | 7.29 |
| Microcrystalline cellulose (Avicel PH-102) | 108.85 | 62.20 |
| Polycarbophil | 50.00 | 28.57 |
| Aspartame | 0.15 | 0.09 |
| Cherry Flavor | 0.25 | 0.14 |
| Stearic acid | 3.00 | 1.71 |
| Uncoated Tablet Weight (mg) | 175.00 | 100.00 |

### Example 21: Composition 21

The composition 21 & 22 was manufactured using Polycarbophil, Hydroxypropyl Methyl Cellulose, Carbopol 974P as polymer using dry mixing technique.

| Ingredients | mg/unit | % w/w |
|---|---|---|
| CLX 156 | 12.750 | 8.50 |
| Lactose Monohydrate | 72.375 | 48.25 |
| Maize Starch | 22.500 | 15.00 |
| Polycarbophil | 3.125 | 2.08 |
| Hydroxypropyl Methylcellulose (Methocel K15M CR) | 26.250 | 17.50 |
| Carbopol 974P | 9.375 | 6.25 |
| Colloidal Silicon dioxide | 1.250 | 0.83 |
| Talc | 1.500 | 1.00 |
| Magnesium Stearate | 0.875 | 0.58 |
| Uncoated Tablet Weight (mg) | 150.00 | 100.00 |

### Example 22: Composition 22

| Ingredients | mg/unit | % w/w |
|---|---|---|
| CLX 156 | 12.75 | 7.29 |
| Microcrystalline Cellulose | 58.85 | 33.63 |
| Methocel K15M CR | 82.50 | 47.14 |
| Carbopol 974P | 4.38 | 2.50 |
| Polycarbophil | 13.13 | 7.50 |
| Aspartame | 0.15 | 0.09 |
| Cherry Flavor | 0.25 | 0.14 |
| Stearic Acid | 3.00 | 1.71 |
| Uncoated Tablet Weight (mg) | 175.00 | 100.00 |

### Example 23: Composition 23

The composition 23 was manufactured using Polycarbophil, Sodium CMC, Carbopol 974P as polymer using dry mixing technique.

| Ingredients | mg/unit | % w/w |
|---|---|---|
| CLX 156 | 12.75 | 8.50 |
| Microcrystalline Cellulose | 73.85 | 49.23 |
| Blanose (Sodium CMC) | 45.00 | 30.00 |
| Carbopol 974P | 3.75 | 2.50 |
| Polycarbophil | 11.25 | 7.50 |
| Aspartame | 0.15 | 0.10 |
| Cherry Flavor | 0.25 | 0.17 |
| Stearic Acid | 3.00 | 2.00 |
| Uncoated Tablet Weight (mg) | 150.00 | 100.00 |

### Example 24: Composition 24

The composition 24 was manufactured using Hydroxypropyl Methyl Cellulose as polymer with Anti-Oxidants using dry mixing technique.

| Ingredients | mg/unit | % w/w |
|---|---|---|
| CLX 156 | 17.00 | 9.71 |
| Pharmacel - 112 (Microcrystalline Cellulose) | 54.30 | 31.03 |
| Methocel K15M CR | 100.00 | 57.14 |
| Butylated Hydroxyanisole | 0.10 | 0.06 |
| Butylated Hydroxytoluene | 0.20 | 0.11 |
| Aspartame | 0.15 | 0.09 |
| Cherry Flavor | 0.25 | 0.14 |
| Stearic Acid | 3.00 | 1.71 |
| Uncoated Tablet Weight (mg) | 175.00 | 100.00 |

### Example 25: Composition 25

The composition 25 was manufactured using Sodium CMC as polymer with Anti-Oxidants using Dry mixing technique.

| Ingredients | mg/unit | % w/w |
|---|---|---|
| CLX 156 | 17.00 | 11.33 |
| Pharmacel - 112 (Microcrystalline Cellulose) | 79.30 | 52.87 |
| Blanose 7H4XFPH (Sodium CMC) | 50.00 | 33.33 |
| Butylated Hydroxyanisole | 0.10 | 0.07 |
| Butylated Hydroxytoluene | 0.20 | 0.13 |
| Aspartame | 0.15 | 0.10 |
| Cherry Flavor | 0.25 | 0.17 |
| Stearic Acid | 3.00 | 2.00 |
| Uncoated Tablet Weight (mg) | 150.00 | 100.00 |

### Example 26: Composition 26

The composition 26 was manufactured using Carbopol 974P as polymer with Anti-Oxidants using Dry mixing technique.

| Ingredients | mg/unit | % w/w |
|---|---|---|
| CLX 156 | 17.00 | 6.77 |
| Pharmacel - 112 (Microcrystalline Cellulose) | 139.30 | 55.50 |
| Carbopol 974P | 90.00 | 35.86 |
| Butylated Hydroxyanisole | 0.10 | 0.04 |
| Butylated Hydroxytoluene | 0.20 | 0.08 |
| Colloidal Silicon Dioxide | 1.00 | 0.40 |
| Aspartame | 0.15 | 0.06 |
| Cherry Flavor | 0.25 | 0.10 |
| Stearic Acid | 3.00 | 1.20 |
| Uncoated Tablet Weight (mg) | 251.00 | 100.00 |

### Example A: Mucoadhesive Test

Objective: To perform Mucoadhesion study for Buccal Tablets and evaluate the adhesive property.

### Batches Evaluated

The following batches were evaluated for adhesion strength. The details of batches manufactured are given below.

| S. No. | Batch Number | Polymers studied |
|---|---|---|
| 1 | AC156C0121005 | Carbomer |
| 2 | AC156C0121009 | Sodium CMC |
| 3 | AC156C0121017 | Methocel K15M CR |
| 4 | AC156C0121018 | Methocel K15M CR, Carbopol 974P, |
| | | Polycarbophil |
| 5 | AC156C0121019 | Methocel K15M CR, Carbomer, Carbopol |
| 6 | AC156C0121020 | Sodium CMC, Carbomer, Carbopol |

Requirements for Test: Texture Analyzer equipment, Buccal or Intestinal Mucosa (Sheep or Pork), Cyanoacrylate Tape, Cutter, Tray, Simulated Salivary Fluid, Test tablets.

### Procedure:

A part of sheep intestinal mucosa was dissected and placed on petri dish.
The tablet to be evaluated was fixed on probe of equipment using cyanoacrylate tape
The test was performed in 3 stages.

### Stage-1 Downward movement of probe

Upon starting the process, the probe moves downward towards the mucosal layer at a constant speed until the tablet touch the mucosal layer. The speed of movement of probe for the test performed was 6 mm/min.

### Stage-2 Contact of Tablet with Mucosa The tablet was kept in contact with mucosal layer for 180 seconds (3 minutes) with a force of 2 N applied on the mucosal layer.

### Stage-3 Upward Movement of Probe

After the contact time is completed, the probe was moved upward at a constant speed (of 6 mm/min) till the mucosal layer is detached from tablet surface.

The force of detachment was recorded, and the adhesion force of the tablet was recorded by software. The test was performed on 3 tablets/batch and average adhesion strength of tablets was given as the Mucoadhesion strength.

Results: The results of adhesion strength of tablets performed are given below,

| | Batch Number | | | | | | | |
|---|---|---|---|---|---|---|---|---|
| | #005 | | #009 | | #014 | | #017 | |
| | Polymer Used | | | | | | | |
| | Carbopol 974P | | Sodium CMC | | Polycarbophil | | Methocel K15M CR | |
| | Parameters | | | | | | | |
| | | Adhesive | | Adhesive | | Adhesive | | Adhesive |
| | Adhesiveness | Force | Adhesiveness | Force | Adhesiveness | Force | Adhesiveness | Force |
| Sample - 1 | −0.0007 | −1.1361 | 0.00892 | 0.0036 | 0.00899 | −0.0999 | 0.01136 | −0.0033 |
| Sample - 2 | −0.0010 | −0.4372 | 0.00871 | −0.0075 | −0.0003 | −0.3236 | 0.00790 | −0.0026 |
| Sample - 3 | −0.0006 | −0.6847 | −0.0002 | −0.0787 | −0.0002 | −0.3649 | Not | Not |
| | | | | | | | Performed | Performed |
| Average | −0.0008 | −0.7527 | 0.00882 | −0.0056 | −0.00283 | −0.2628 | 0.00963 | −0.003 |
| Maximum | −0.0006 | −0.4372 | 0.00892 | −0.0036 | 0.00899 | −0.0999 | 0.01136 | −0.0026 |
| Minimum | −0.0010 | −1.1361 | 0.0087 | −0.0075 | −0.0003 | −0.3649 | 0.0079 | −0.0033 |

| | Batch Number | | | | | |
|---|---|---|---|---|---|---|
| | #018 | | #019 | | #020 | |
| | Polymer Used | | | | | |
| | Methocel K15M CR, | | Methocel K15M CR, | | Sodium CMC, | |
| | Carbopol 974P, | | Carbopol 974P, | | Carbopol 974P, | |
| | Polycarbophil | | Polycarbophil | | Polycarbophil | |
| | Parameters | | | | | |
| | | Adhesive | | Adhesive | | Adhesive |
| | Adhesiveness | Force | Adhesiveness | Force | Adhesiveness | Force |
| Sample - 1 | −0.0001 | −0.2418 | −0.0002 | −0.3259 | 0.00727 | −0.0001 |
| Sample - 2 | −0.0002 | −0.1959 | −0.0005 | −0.4089 | 0.00818 | −0.0002 |
| Sample - 3 | −0.0001 | −0.5478 | 0.00001 | −0.0010 | Not | Not |
| | | | | | Performed | Performed |
| Average | −0.00013 | −0.3285 | −0.00023 | −0.24527 | 0.00773 | −0.0002 |
| Maximum | −0.0001 | −0.1959 | 0.00001 | −0.001 | 0.00818 | −0.0001 |
| Minimum | −0.0002 | −0.5478 | −0.0005 | −0.4089 | 0.00727 | −0.0002 |

Conclusion: Batches manufactured with Sodium CMC had a better Mucoadhesion strength than other polymer batches.

### Example B: Study to Investigate the Effects of CLX 156 on H₂O₂-Induced Oxidative Stress in A-253, Human Submaxillary Salivary Gland (HSG) Cell Line

Objective: The objective of this study was to investigate the effect of the test compound (CLX 156), on H₂O₂-induced oxidative stress in human submaxillary gland (HSG) cell line; A-253 cell line, in comparison with pilocarpine hydrochloride.

Method: A-253 human salivary gland cell line was cultured in McCoy's 5a Medium Modified (ATCC) with 10% HI-FBS according to manufacturer's guidelines at 37° C. in the presence of 5% CO 2 in a humified incubator.

The cells were sub-cultured using Trypsin-EDTA and 10,000 cells seeded into each 48 well cell culture plates with a media volume of 500 μl. The cells were allowed to grow for 48 hours. After 48 hours of culture, the cells were pre-incubated for 1 hour with the positive, reference and test compounds at the desired concentrations (see table 1) followed by the addition of 1 mM H₂O₂ to the culture media (without the removal of test compounds) to induce oxidative stress. The incubation was continued for 24 hours.

After 24 hours of treatment with H₂O₂, the cells were used to assess iROS as described below.

This is a cell-based assay for measuring hydroxyl, peroxyl, or other reactive oxygen species activity within a cell. The assay employs the cell-permeable fluorogenic probe 2′, 7′-Dichlorodihydrofluorescin diacetate (DCFH-DA). In brief, DCFH-DA is diffused into cells and is deacetylated by cellular esterases to non-fluorescent 2′,7′-Dichlorodihydrofluorescin (DCFH), which is rapidly oxidized to highly fluorescent 2′,7′-Dichlorodihydrofluorescein (DCF) by ROS. The fluorescence intensity is proportional to the ROS levels within the cell cytosol. OxiSelect˜ Intracellular ROS Assay Kit was used according to the manufacturer's guidelines (Cell Biolabs). In short, at Day 3 the cells were washed several times with PBS and then treated with 1X DCFH-DA in media and incubated for 30-60 minutes. After DCFH-DA treatment, the oxidative stress was induced by adding 1 mM H₂O₂ and the fluorescence signals measured after 24 hours using a plate reader.

TABLE 1
iROS assay - normalized data

| | | | | Average | | | | |
|---|---|---|---|---|---|---|---|---|
| Treatment | Concentration | | Stimulation | raw value | iROS assay | | | Average |
| Unstimulated | None | | None | 803.33 | 1.0000 | | | 1.0000 |
| cells | | | | | | | | |
| DMSO | 0.5% | v/v | None | | 0.9934 | 0.9797 | 1.1353 | 1.0361 |
| H₂O₂ | 1 | mM | None | | 7.5062 | 7.6680 | 7.5062 | 7.5602 |
| Pilocarpine | 3 | μM | H₂O₂(1 mM) | | 7.5560 | 7.7676 | 8.2656 | 7.8631 |
| HCl | 10 | μM | H₂O₂(1 mM) | | 6.6722 | 6.6224 | 7.1577 | 6.8174 |
| | 30 | μM | H₂O₂(1 mM) | | 6.7593 | 6.2988 | 7.2448 | 6.7676 |
| | 100 | μM | H₂O₂(1 mM) | | 7.2199 | 7.0581 | 6.6473 | 6.9751 |
| | 300 | μM | H₂O₂(1 mM) | | 6.8340 | 6.6971 | 7.4813 | 7.0041 |
| | 1000 | μM | H₂O₂(1 mM) | | 6.7469 | 6.6846 | 7.0705 | 6.8340 |
| CLX 156 | 3 | μM | H₂O₂(1 mM) | | 6.2116 | 6.0622 | 5.9502 | 6.0747 |
| | 10 | μM | H₂O₂(1 mM) | | 5.3278 | 5.2780 | 5.7386 | 5.4481 |
| | 30 | μM | H₂O₂(1 mM) | | 5.5145 | 5.7137 | 5.8631 | 5.6971 |
| | 100 | μM | H₂O₂(1 mM) | | 5.9627 | 5.5768 | 5.5643 | 5.7012 |
| | 300 | μM | H₂O₂(1 mM) | | 4.7054 | 5.2656 | 4.9544 | 4.9751 |
| | 1000 | μM | H₂O₂(1 mM) | | 3.5353 | 3.3237 | 3.6598 | 3.5062 |

TABLE 2
iROS assay - normalised data vs H₂O₂

| | | | | Average | | | | |
|---|---|---|---|---|---|---|---|---|
| Treatment | Concentration | | Stimulation | raw value | iROS assay | | | Average |
| H₂O₂ | 1 | mM | None | 6073.33 | 1.0000 | | | 1.0000 |
| Pilocarpine | 3 | μM | H₂O₂(1 mM) | | 0.9995 | 1.0274 | 1.0933 | 1.0401 |
| HCl | 10 | μM | H₂O₂(1 mM) | | 0.8825 | 0.8760 | 0.9468 | 0.9018 |
| | 30 | μM | H₂O₂(1 mM) | | 0.8941 | 0.8332 | 0.9583 | 0.8952 |
| | 100 | μM | H₂O₂(1 mM) | | 0.9550 | 0.9336 | 0.8793 | 0.9226 |
| | 300 | μM | H₂O₂(1 mM) | | 0.9040 | 0.8858 | 0.9896 | 0.9265 |
| | 1000 | μM | H₂O₂(1 mM) | | 0.8924 | 0.8842 | 0.9352 | 0.9040 |
| | 3 | μM | H₂O₂(1 mM) | | 0.8216 | 0.8019 | 0.7870 | 0.8035 |
| | 10 | μM | H₂O₂(1 mM) | | 0.7047 | 0.6981 | 0.7591 | 0.7206 |
| | 30 | μM | H₂O₂(1 mM) | | 0.7294 | 0.7558 | 0.7755 | 0.7536 |
| | 100 | μM | H₂O₂(1 mM) | | 0.7887 | 0.7377 | 0.7360 | 0.7541 |
| | 300 | μM | H₂O₂(1 mM) | | 0.6224 | 0.6965 | 0.6553 | 0.6581 |
| | 1000 | μM | H₂O₂ (1 mM) | | 0.4676 | 0.4396 | 0.4841 | 0.4638 |

### INFERENCE

CLX 156 significantly inhibited the iROS induced by H₂O₂ in A-253 cells in a concentration dependent manner.
The effect observed with Pilocarpine HCl was comparatively less in magnitude and showed no clear dose response.
CLX 156 is superior to Pilocarpine HCL in inhibiting the iROS induced by H₂O₂ in A-253 salivary gland cell culture model.
IC50 for pilocarpine-R-lipoate was 551.7 μM for Intracellular ROS (iROS) inhibition.

What is claimed is:

1. A pharmaceutical composition for oral administration comprising pilocarpine R-(+)-lipoate or its polymorphs, enantiomers, isomers; and its pharmaceutically acceptable salts thereof, wherein the pilocarpine R-(+)-lipocate is formulated in an amount of 1% to 70% w/w of the composition; and (ii) one or more excipients in an amount of 30-99% w/w of the composition.

2. (canceled)

3. The pharmaceutical composition of claim 2, wherein the pilocarpine-R-(+)-lipoate is in the range of 0.1 mg to 100 mg.

4. The pharmaceutical composition of claim 2, wherein the one or more excipients are selected from a group consisting of a diluent, a lubricant, a disintegrant, a polymer, a flavoring agent, a binder, a sweeting agent, a glidant, an antioxidant, a coating material, mixtures thereof.

5. The pharmaceutical composition of claim 4, wherein the diluent is selected from a group consisting of lactose, spray dried lactose, lactose monohydrate, lactose hydrous, lactose anhydrous, starches, maize starches, or partially pregelatinized starches, sucrose, magnesium stearate, glucose, micro crystalline cellulose, Polyvinylpyrrolidone, mannitol, sorbitol, dibasic calcium phosphate dehydrate, calcium sulphate dehydrate, calcium carbonate, and mixtures thereof.

6. The pharmaceutical composition of claim 4, wherein the disintegrant is selected from a group consisting of a crosslinked polymer, crosslinked sodium carboxymethylcellulose microcrystalline cellulose, alginates, modified starches, mixtures thereof.

7. The pharmaceutical composition of claim 4, wherein the polymer is selected from a group consisting of carbomers, polycarbophil, pemulen polymers, starch, modified cellulose, crystalline cellulose, microcrystalline cellulose, carboxymethyl cellulose, sodium carboxymethylcellulose, an acrylic acid copolymer, methyl vinyl ether copolymer with maleic anhydride, hydroxypropyl methylcellulose, polyglycolic acid, and mixtures thereof.

8. The pharmaceutical composition of claim 4, wherein the flavoring agent is selected from a group consisting of clove oil, citric syrup, glycerin, rose oil, orange oil, menthol, cherry, and mixtures thereof.

9. The pharmaceutical composition of claim 4, wherein the binder is selected from a group consisting of saccharides, starches, cornstarch, cellulose, methyl cellulose, modified cellulose, microcrystalline cellulose, cellulose ethers, hydroxypropyl cellulose, sugar alcohols, xylitol, sorbitol, or mannitol, protein, gelatin, synthetic polymers, polyvinyl pyrrolidone, polyethylene glycol, and mixtures thereof.

10. The pharmaceutical composition of claim 4, wherein the sweeting agent is selected from a group consisting of sucrose, liquid glucose, glycerol, sorbitol, saccharin sodium, aspartame, and mixtures thereof.

11. The pharmaceutical composition of claim 4, wherein the glidant is selected from a group consisting of magnesium stearate, fumed silica, starch, talc, and mixtures thereof.

12. The pharmaceutical composition of claim 4, wherein the antioxidant is selected from a group consisting of butylated hydroxyanisole, butylated hydroxytoluene, sodium metabisulfite, propyl gallate, cysteine, ascorbic acid, and mixtures thereof.

13. The pharmaceutical composition of claim 4, wherein the a coating material is selected from a group consisting of sugar, polymers, polysaccharides, moisture barrier coating material, cellulosic polymers, vinyl derivatives, hydroxypropyl cellulose, Hydroxyethyl cellulose microcrystalline cellulose, derivatives cellulose, alkylated cellulose, ethyl cellulose, propyl cellulose, hydroxylpropyl cellulose, polysaccharide, hydroxypropyl methylcellulose, carboxymethylcellulose, maltodextrin, sucrose, modified starch, of alginic acid, soluble gums, carrageenan, polyvinylpyrrolidone, polyvinylpolypyrrolidone, and Opadry film coating system.

14. The pharmaceutical composition of claim 4, wherein the coating of the composition is selected from a group consisting of sugar coatings, film coatings, gelatin coatings, enteric coatings, compression coatings, and immediate-release film coatings.

15. The pharmaceutical composition of claim 1, further comprising a modified release composition, wherein the modified release composition is formulated into mucoadhesive buccal tablet, lozenge, oral patch, oral film, buccal patch, oral spray, oral solution, oral gel, sub-lingual tablet, mucoadhesive patch or film or transdermal patch.

16. The pharmaceutical composition of claim 15, wherein the modified release is controlled by polymers and pharmaceutically acceptable excipients to deliver the pharmaceutical composition by extended release, sustained release, or delayed release.
